(19) United States
(12) Patent Application Publication
(10) Pub. No.: US 2008/0234830 A1
(43) Pub. Date: Sep. 25, 2008

(54) Femoral Head Having A Spherical Backside Surface

(75) Inventors: Troy W. Hershberger, Winona Lake, IN (US); Kurt Schmidt, Warsaw, IN (US)

Correspondence Address: HARNESS, DICKEY & PIERCE, P.L.C., P.O. BOX 828, BLOOMFIELD HILLS, MI, 48303, US

(73) Assignee: BIOMET MANUFACTURING CORP., Warsaw, IN (US)

(21) Appl. No.: 12/040,314

(22) Filed: Feb. 29, 2008

Related U.S. Application Data
(60) Provisional application No. 60/892,433, filed on Mar. 1, 2007.

(51) Int. Cl. A61F 2/32; A61F 2/38

(52) U.S. Cl. 623/22.15; 623/20.36

(57) ABSTRACT

A prosthesis member or assembly can be implanted in a portion of the anatomy. The prosthesis member, with various portions, defines a selected amount of a sphere to articulate or engage selected portions of the anatomy.

### CROSS-REFERENCE TO RELATED APPLICATIONS

This application claims the benefit of U.S. Provisional Application No. 60/892,433, filed on Mar. 1, 2007. The disclosure of the above application is incorporated herein by reference.

### FIELD

The present disclosure relates to a prosthesis, and particularly to a generally spherical or majority spherical prosthetic member for an articulation.

### BACKGROUND

The statements in this section merely provide background information related to the present disclosure and may not constitute prior art.

In an anatomy, a prosthesis can articulate with a second prosthetic portion or a natural anatomy. It can be selected to have the prosthesis substantially mimic a natural articulation. A natural articulation may allow or require a substantially large range of motion. Therefore, it may be desirable to provide a prosthesis that includes a surface that can articulate through all or substantially all of the natural articulation motion.

### SUMMARY

A prosthesis member can be positioned in an anatomy to substantially allow for a natural motion of an articulating joint. For example, a femoral head, generally at a proximal end of the femur, can be provided to articulate with an acetabulum or an acetabular prosthesis. The femoral head prosthesis can be provided as at least a part of a sphere. In addition, or as an alternative, substantially all of a spherical portion can be provided. Although it will be understood that the substantially spherical head can include a portion that allows for interconnection with a stem or neck portion, the remaining portion of the surface can define substantially an entire sphere for articulation. The prosthesis head of the femur can be provided as one member or as a modular portion to allow for a large range of articulation with an anatomy or prosthesis member.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a partial cross-sectional view of an assembled prosthesis, according to various embodiments;

FIG. 2 is a partial cross-sectional view of an assembled prosthesis, according to various embodiments;

FIG. 3 is a partial cross-sectional view of an assembled prosthesis, according to various embodiments;

FIG. 4 is a partial cross-sectional view of an assembled prosthesis, according to various embodiments;

FIG. 5 is a partial cross-sectional exploded view of a prosthesis, according to various embodiments;

FIG. 6 is a partial cross-sectional exploded view of a prosthesis, according to various embodiments;

FIG. 7 is a partial cross-sectional view of an assembled prosthesis, according to various embodiments;

FIG. 8 is a partial cross-sectional view of an assembled prosthesis, according to various embodiments; and

FIG. 9 is a detail environmental view of a prosthesis, according to various embodiments.

### DETAILED DESCRIPTION

The following description is merely exemplary in nature and is not intended to limit the present disclosure, application, or uses.

A prosthesis can be positioned at various portions of the anatomy, such as a proximal femoral replacement which can also be referred to as a femoral head. Femoral head prostheses can include the M2a Magnum™ sold by Biomet, Inc. of Warsaw, Ind.

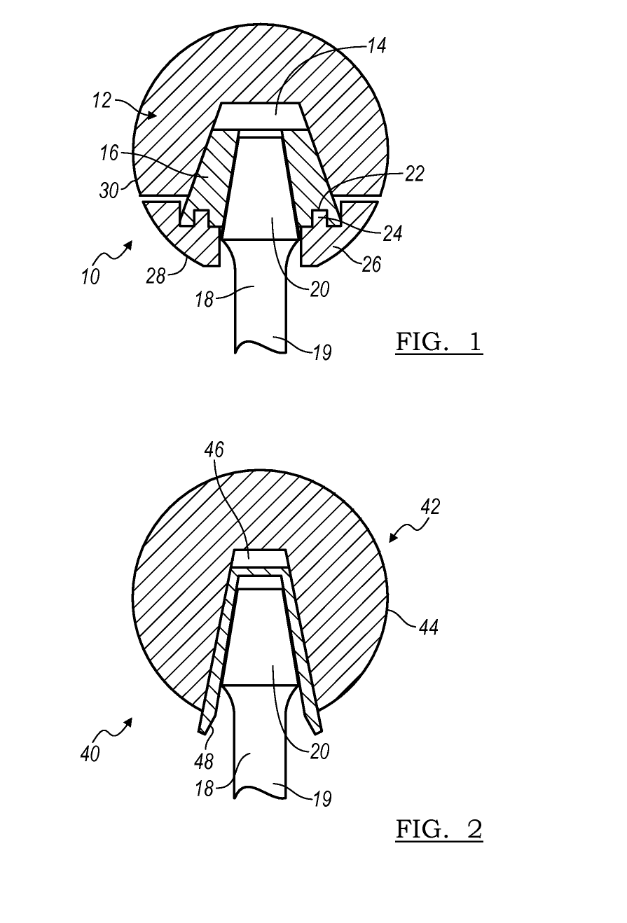

With reference to FIG. 1, an exemplary prosthesis is illustrated. A femoral head prosthesis 10 can include an exterior head or articulation surface 12. The exterior head 12 can include an open or hollow interior 14. An adapter 16 can be provided to interconnect the head portion 12 with a stem or neck portion 18. The neck portion 18 can include a male taper 20 that can engage the adapter 16 that engages the head 12. In this way, the head 12 can be interconnected with the neck or stem 18. As is understood, the neck 18 can extend to a stem portion 19 (FIGS. 2 and 9) that can extend into a bone, such as a femur 304 (FIG. 9). The stem portion can extend into an intramedullary canal or other appropriate portion of the bone or can be affixed to an exterior of the bone.

The adapter 16 can include a shield connection portion 22. The shield connection portion 22 can include a bore or multiple bores to receive an adapter connection portion or finger 24. The adapter connection portion 24 can include a finger or extension to extend into the bores 22. In addition, the shield connection portion can include a dovetail lock, a finger and ledge engagement, or other appropriate engagements.

The fingers 24 can extend into the bores 22 and provide any appropriate connection. For example, a snap connection, a taper connection, a welded connection, an adhesive connection, or any appropriate connection can be provided. Nevertheless, the finger 24 can extend to and/or into the bore 22 to hold a shield 26 relative to the adapter 16 and the head 12. It will also be understood that the bores 22 can be provided in any appropriate shape such cylindrical, polygonal, or other appropriate shape. In this way, the shield 26 can include an exterior surface 28 that, when interconnected with the adapter 16, can substantially continue the exterior surface 30 of the head 12 to have a substantially or majority spherical surface that is substantially only interrupted by an opening to receive the neck portion 18 of the stem. As discussed and illustrated further herein, the shield can be used to reduce impingement or abrasion of selected anatomic features.

With reference to FIG. 2, a prosthesis 40 is illustrated. The prosthesis 40 can include portions that are similar to the prosthesis 10 illustrated in FIG. 1. For example, the prosthesis 40 can include a neck 18 that extends from a stem 19. As discussed above, the stem 19 can extend into any appropriate portion of the bone, such as an intramedullary canal a femur 304 or can be provided to extend around or be connected to an exterior of the femur.

The prosthesis 40 can also include a head portion 42 that includes an articulating surface 44. The articulating surface 44 can extend to substantially define a spherical surface. For example, the articulating surface 44 can extend about 280 to about 360 degrees, including about 340 degrees. An opening or hollow portion 46 can be defined by the head portion 42. An adapter sleeve or member 48 can extend into the hollow or opening portion 46 and includes a female taper portion to engage the taper 20 of the neck.

The adapter 48 can be provided in a plurality of sizes, offsets, lengths, or the like. The adapter 48 can fit within the opening 46 to allow for interconnection of the head 42 with the neck 18 or the taper 20. For example, a set of adapters 48 can be provided that allows for a specific location of the head 42 relative to the stem 19. The location can include an axial distance, an offset, etc. Further, the adapters can be provided to have different offsets, lengths, anatomical positions, or the like.

A plurality of the heads 42 can be provided to engage one neck portion 18 or a plurality of neck portions 18 can be provided to engage a single head portion 42 with provision of the appropriate adapter. Also, a single adapter 48 can be provided to interconnect one of a plurality of heads with the neck 18. For example, the adapter 18 can be used to interconnect one of a plurality of the heads 42 where each includes a different diameter.

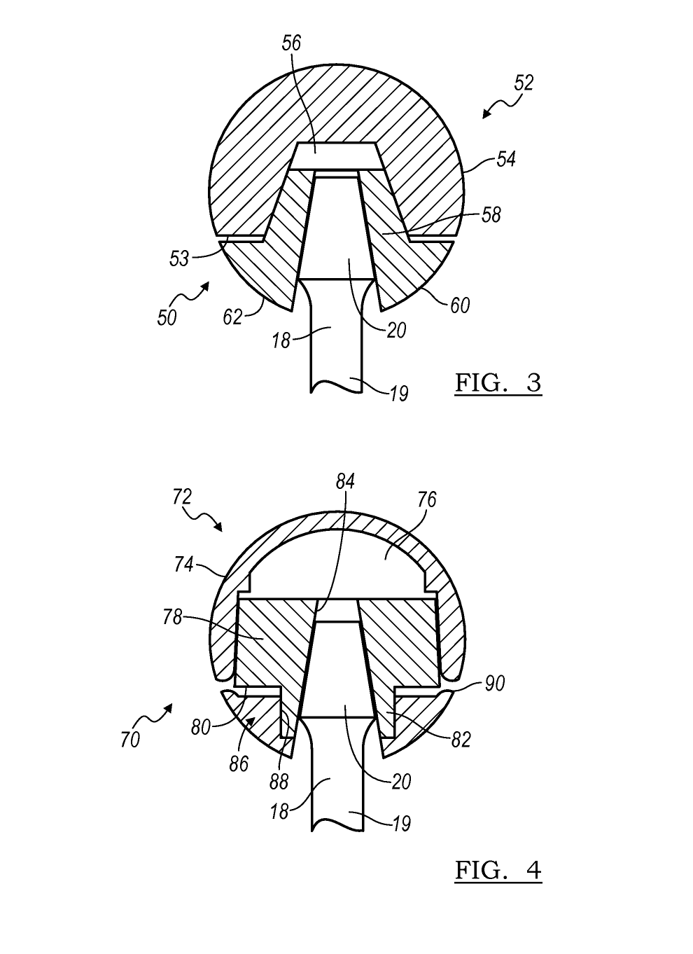

With reference to FIG. 3, a prosthesis 50 is illustrated. The prosthesis 50 can include a neck 18, a stem 19, and a taper connection 20, similar to those described above. Further, the prosthesis 50 can include a prosthetic femoral head 52 that defines an articulating surface 54. The head 52 can define an internal opening or void region 56 and an adapter 58 can be provided to interconnect the head 52 and the taper 20.

The adapter 58 can include an extending or completing region 60. The completing region 60 can define an articulating or articular completion surface 62. The surface 62 can include an arc or surface contour that is substantially similar to the arc or surface contour of the articulating surface 54 of the head 52. In this way, a substantially more complete or completely spherical surface can be provided with the prosthesis 50. The adapter 58 can be provided in a plurality of sizes, lengths, offsets, dimensions, and the like for interconnection with one or a plurality of heads 52 or the necks 18. The adapter 58 can be provided with various features and configurations similar to those discussed above with respect to the adapter 48 to meet similar reasons and purposes.

The completion portion 60 can extend from or engage relative to a bottom or flat portion 53 of the head 52. The articular surface 54 of the head 52 can be generally spherical or arcuate over a majority of its portion and include the flat portion 53. The completion portion 60 can be positioned near the substantially flat portion 53. The completion portion 60 of the adapter 58 can be provided to include an edge or shoulder that engages the flat portion 53 so that the articular surface remains continuous to and over the completion portion 60.

With reference to FIG. 4, a prosthesis 70 is illustrated. The prosthesis 70 can include a neck portion 18 that defines a taper portion 20, as discussed above. Again, the neck portion 18 can extend from a stem (not illustrated in FIG. 4, but similar to that illustrated in FIGS. 2 and 9) that can interconnect with the selected portion of the anatomy.

The prosthesis 70 can also include a head portion 72 that defines an exterior articular surface 74 and an internal void or opening 76. The articular surface 74 can end near an adapter 78 that includes a substantially flat portion 80. The adapter 78 can define a male taper portion 82 that can extend around the taper 20 of the neck 18. The adapter 78 can also define a female taper 84 within the adapter 78 to engage the taper 20 of the neck 18.

A guard or completion portion 86 can define an internal or female taper 88 to engage the male taper 82 of the adapter 78. The guard 86 can include an exterior surface 90 that can include an arc substantially congruent or cooperating with the exterior surface 74 of the head 72. In this way, the prosthesis 70 can include a substantially completely spherical head portion that can articulate with a selected portion of the anatomy. Nevertheless, the modular portions can be interconnected during the procedure, such as pre-operatively or intra-operatively, to provide for the substantially spherical articular portion. In addition, the guard 86, adapter 78, and the head 72 can be provided in various configurations, sizes, etc., for selection and interconnection during an operative procedure.

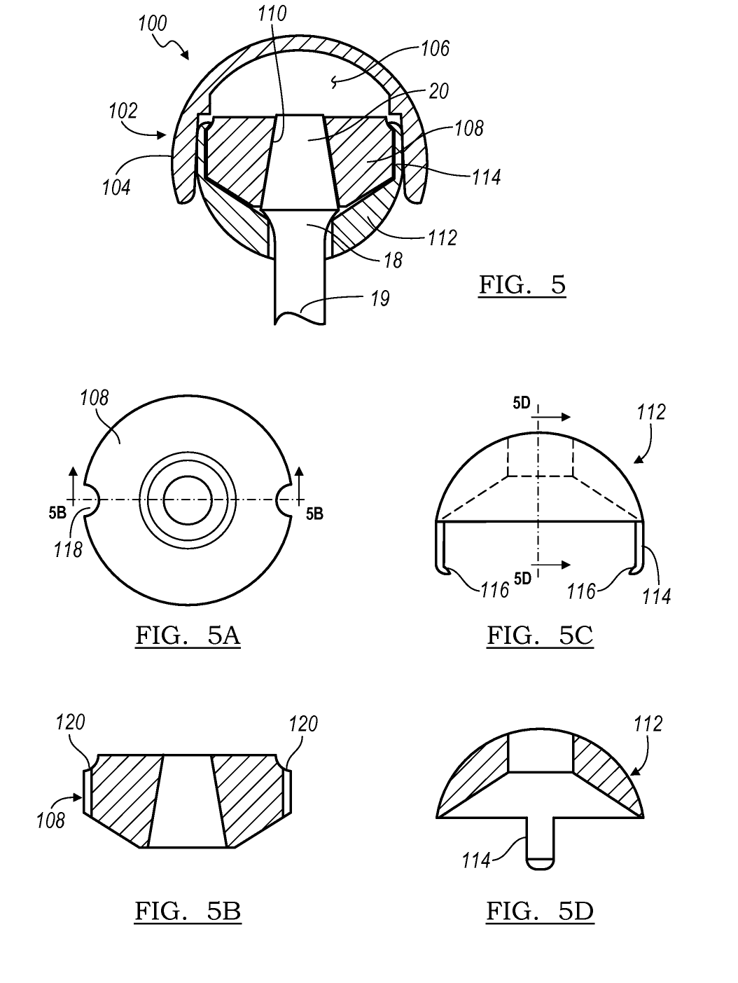

With reference to FIG. 5, a prosthesis 100 is illustrated. The prosthesis 100 can include a neck 18 and a taper portion 20 as discussed above. The prosthesis 100 can also include a head portion 102 that includes an articular surface 104. An internal void or opening 106 can also be provided within the head portion 102. An adapter 108 can be provided to interconnect, such as with a taper, to head 102 to hold the head 102 in a selected position. The adapter 108 can also define an internal taper 110 to engage the taper 20 of the neck.

A guard portion or connection second portion 112 can be provided that includes an extension arm or grasping portion 114. Grasping portion 114 can include a connection tip or finger 116.

The adapter 108 can include a notch 118 to receive the extension portion 114 and an upper ledge or ledge portion 120 to engage or receive the finger 116. The ledge 120 can provide a surface for the finger 116 to engage. The finger 116 can be biased on the ledge 120 by the extension arm 114. In this way, the guard portion 112 can extend around the neck portion 18 including the extension arm 114 to extend in the notches 118. The notches 118 allow the guard portion 112 to engage the adapter 108 while allowing the head 102 to engage the adapter 108 with a taper engagement or locking portion.

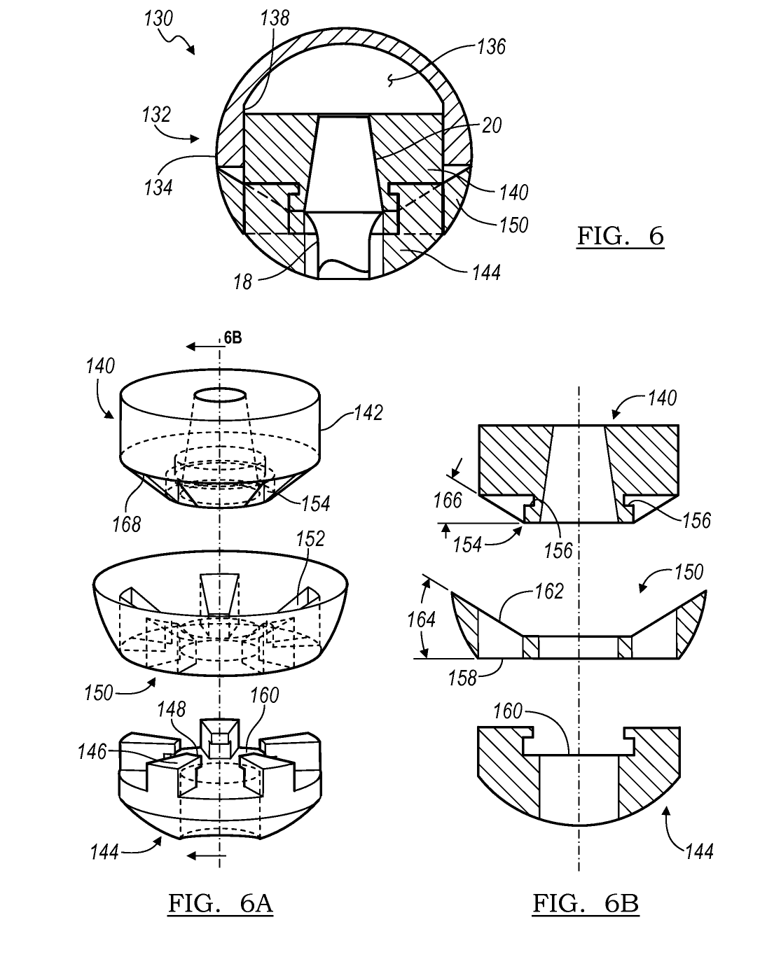

With reference to FIG. 6, a prosthesis 130 is illustrated. The prosthesis 130 can include the neck portion 18 and the taper connection portion 20 as discussed above. Further, the prosthesis 130 can include a head portion 132 that includes an articular or spherical surface 134. Further, the head portion 132 can define an internal opening or void 136. It will be understood that the head portion 132 can include a substantially spherical or completely spherical exterior portion, such as the head portion disclosed and discussed above. The cut away view is for clarity of the current discussion.

The opening 136 can define an internal or female taper 138 which can engage with an adapter 140. The adapter 140 can include an exterior surface 142 that includes a taper to engage with the taper 138 of the head 132. A guard portion 144 can be provided to substantially complete or allow for a greater spherical surface to be defined by the prosthesis 130.

The guard portion 144 can include extensions or projections 146 that include fingers 148. Any appropriate number of the projections 146 and fingers 148 can be provided. The projections 146 extend from a surface of the guard portion 144 and the fingers 148 can extend from the projections 146 a distance from the surface,

A plate 150, that can include a conical or circular portion, can be provided that includes passages 152. The passages 152 can allow the projections 146 to pass through or engage a receiving portion 154 in the adapter 140. The receiving portions 154 can include a ledge 156 to receive the fingers 148 for an engagement.

The conical plate 150 can include a bottom surface 158 that is substantially flat. The bottom surface 158 can engage a top surface 160 of the shield 144. The conical plate 150 can also include a top surface that is angled or includes a slant portion 162. An angle 164 can be defined between the bottom surface 158 and the top surface 162. The angle 164 can be substantially identical or complementary to a second angle 166 defined between a bottom surface of the adapter 140 and an edge or ledge portion 168 of the adapter 140. Therefore, the conical plate 150 can allow for an engagement or mating of the shield portion 144 and the adapter 140. An opening can be defined through the shield 144 and the conical plate 150 to allow the taper 20 to engage the adapter 140 and this allows the adapter to be held relative to the neck 18 and engage the internal taper 138 of the head 132.

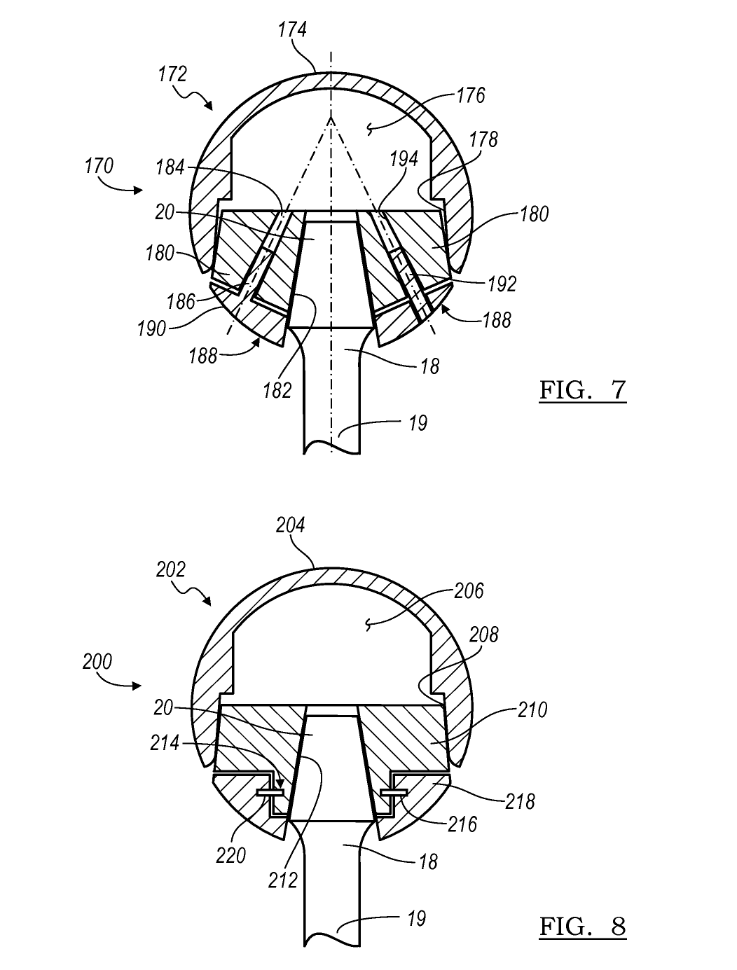

With reference to FIG. 7, a prosthesis 170 is illustrated. The prosthesis 170 can include a neck or trunion 18 as discussed above and includes a taper 20, also discussed above. The prosthesis 170 includes a head portion 172 that can include an arcuate and articulable surface 174. The head 172 can also define an internal void 176 that defines a taper 178. An adapter 180 can engage the taper 178 and also define a female taper 182 to engage the taper 20.

The adapter 180 can also include one or a plurality of passages 184. The passages 184 can receive or engage a finger 186 that extends from a guard portion 188. The guard portion 188 can include or define an exterior surface 190 that can substantially continue the exterior surface 174 of the head portion 172.

The guard portion 188 can also engage or cooperate with a tapered peg or spike 192. The tapered spike 192 can include an exterior surface that can engage a female taper portion 194 in the adapter. Further, the passage 184 and a passage of the taper 194 can be provided at an angle relative to one another to assist in resisting movement of the guard 188 relative to the head 172.

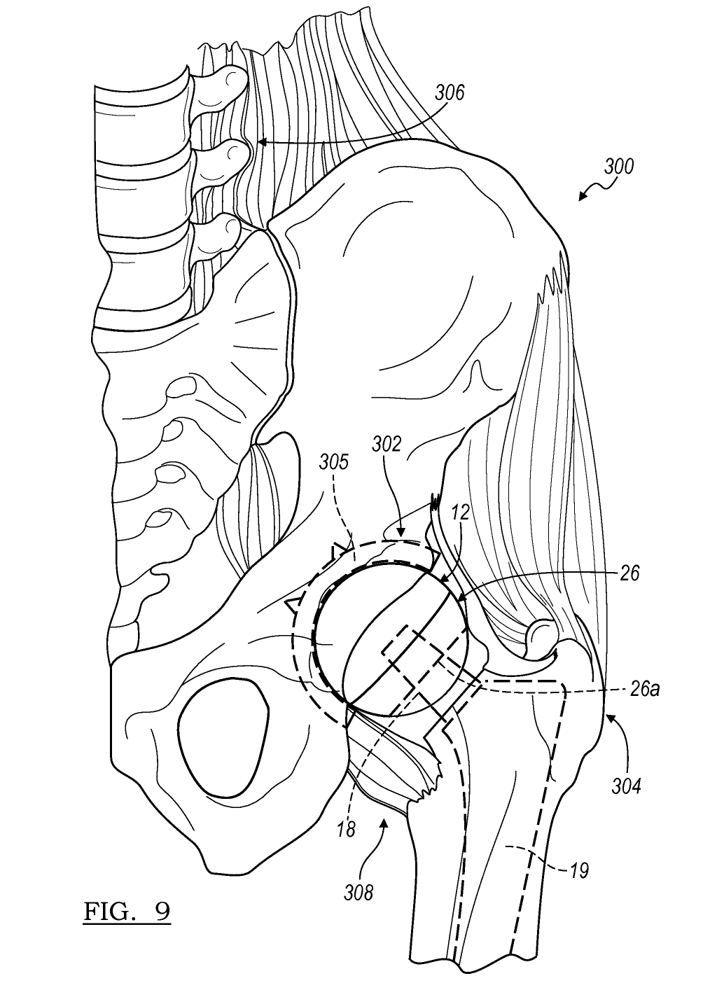

With reference to FIG. 8, a prosthesis 200 is illustrated. The prosthesis 200 can include a neck 18 and taper 20 as discussed above. Further, prosthesis 200 can include a head portion 202 that defines an articular exterior surface 204 that can be substantially arcuate or spherical. Further, an internal void or opening 206 can be defined by the head 202. The opening 206 can define an internal taper 208 with which an adapter 210 can mate with the taper 208. The adapter 210 can also define an internal or female taper 212 to engage the taper 20.

The adapter 210 can also define a ridge or engagement portion 214 to receive or cooperate with a snap or ring lock 216. A guard portion 218 can also define a passage or opening 220 to cooperate with the snap ring or ring lock 216. The ring 216 can be a substantially annular ring that includes an opening to allow it to expand around or be expanded and contract to engage various passages. Moreover, the ring 216 can allow an interconnection of the protector 218 with the adapter 210 to hold the protector 218 axially relative to the adapter 210. Further, an opening defined by the protector 218 can be substantially non-annular to resist rotational motion of the protector 218 relative to the head or other portions of the prosthesis 200. The opening can be square, polygonal, etc.

It will be understood, according to various embodiments, that a guard can be provided for appropriate reasons. If a guard is provided with a femoral head prosthesis it can substantially define a spherical portion with a femoral head to eliminate or minimize the possibility of an edge contacting a selected muscle group or anatomical portion, such as the PSOAS or a tendon that extends from the PSOAS which can also be considered a part of the PSOAS.

With reference to FIG. 9, a prosthesis according to various embodiments can be provided to guard or protect a selected portion of the anatomy. For example, the prosthesis 10, illustrated in FIG. 1, is illustrated in an environmental view. The prosthesis 10 can be provided to replace, in an anatomy 300, a femoral head of a femur 304. The prosthesis 10 can articulate or move relative to an acetabulum 302 of the anatomy 300. The acetabulum can be a natural acetabulum of include an acetabular prosthesis 305. The acetabular prosthesis can be any appropriate prosthesis such as a metal prosthesis with a metal bearing surface. In addition, the prosthesis can include a bearing liner such as a polymer bearing, ceramic bearing, metal bearing, etc. Also, the prosthesis 10, such as the head 12, can be formed of selected metal portions. It will also be understood that the shield 26 can be formed of various materials, such as metal, ceramic, polymer materials, or combinations thereof.

Extending from or near a spinal column 306 of the anatomy can be an anatomical portion 308, such a PSOAS or other muscle groups. PSOAS or other muscle groups can terminate near the femur 304 in a tendon or as the muscle fibers. The prosthesis 10, including the guard or completion portion or shield 26, can protect the anatomical portion 308 from an edge of the head 12. The shield 26 can help ensure that the anatomical portion 308 engages substantially smooth and broad surfaces during articulation of the prosthesis 10. It will be understood, by one skilled in the art, that according to various embodiments shields or guards can be provided.

The shield 26, according to various embodiments, is provided to engage the anatomical portion 308, such as the PSOAS muscle or tendon, rather than engaging a selected portion of the head 12. For example, the head 12 can have various edges or surfaces, such as a substantially flat surface, that can engage the anatomical portion 308. The head 12 can define a spherical surface having a diameter, but at a selected position the spherical surface may terminate and the head can have the flat surface, as illustrated in various embodiments. This can create a void between the head and the femur 304, the neck 20, or other portions. The shield 26, however, can cover or obscure the flat surface of the head 12. The shield 26 can also assist in assuring that the anatomical portion 308 is not pinched or positioned between the head 12 and the acetabulum or selected prosthesis member, such as the acetabular prosthesis 305, at least by filling at least a portion of the void. The shield 26 can fill the void by completing a larger portion of the sphere of the head 12. A separate shield member can be provided for ease of implantation, customization, etc.

The foregoing description is considered illustrative only. Furthermore, it is not desired to limit the scope of the disclosure to the exact construction and process described above. Accordingly, all suitable modifications and equivalents fall within the scope of the disclosure as defined by the claims that follow.

What is claimed is:

1. A prosthesis for replacing a portion of an anatomy, comprising:
an acetabular cup operable to be positioned in an acetabulum of the anatomy;
a femoral head operable to replace a portion of a joint in the anatomy and having a first surface operable to articulate with the acetabular cup and a second surface extending at an angle from the first surface;
a stem operable to extend into the portion of the anatomy;
an engaging member extending from the stem and operable to engage the femoral head; and
a shield extending from the femoral head having a third surface operable to cooperate with the first surface of the femoral head and operable to guard a selected portion of the anatomy from contact with the second surface;
wherein the shield is operable to cover at least a portion of the second surface.

2. The prosthesis of claim 1, wherein the femoral head and the shield are operable to be fixed relative to one another so that the first surface and the third surface are substantially continuous.

3. The prosthesis of claim 1, wherein the femoral head, at a selected cross-section, defines a first portion of a circle and the shield defines a second portion of the circle;
wherein the first portion and the second portion of the circle combined is about 280 degrees to about 360 degrees.

4. The prosthesis of claim 1, wherein the femoral head, at a selected cross-section, defines a substantially arcuate surface having a diameter that terminates at a selected position away from the engaging member defining a void;
wherein the shield includes a surface substantially having the diameter and operable to be positioned in the void.

5. The prosthesis of claim 1, wherein the femoral head comprises a metal, the acetabular cup comprises a metal, and the shield comprises a material selected from the group consisting of polymers, metal, or combinations thereof.

6. The prosthesis of claim 2, further comprising:
an adapter member operable to interconnect with both of the femoral head and the shield to hold the femoral head relative to the shield.

7. The prosthesis of claim 6, wherein the adapter member is further operable to engage the stem to hold the femoral head and the shield relative to the stem.

8. The prosthesis of claim 6, wherein the femoral head includes a bore operable to engage a projection from the shield to interconnect the shield and the adapter member.

9. The prosthesis of claim 1, wherein the shield includes at least three members including;
a first member having a substantially smooth and arcuate exterior surface extending from a first side of a plane and a projection extending from a second side of the plane;
a second member including a bore operable to allow the projection to pass; and
a third member having a bore and an engaging surface to engage the projections;
wherein the third member is operable to engage the femoral head to hold the shield relative to the femoral head; and
wherein the second member is held between the first member and the third member.

10. The prosthesis of claim 6, wherein the adapter member includes a notch extending towards a center of the adapter member from an exterior surface of the adapter member;
wherein the shield includes a projection extending from a first position wherein the third surface extends opposite the projection from the first position;
wherein the projection extends through the notch and engages the third portion to hold the second portion relative to the third portion.

11. The prosthesis of claim 6, further comprising:
a locking member;
wherein the shield includes a first groove and the adapter member includes a second groove;
wherein the locking member engages the first groove and the second groove to interconnect the shield and the adapter member.

12. The prosthesis of claim 6, wherein the adapter is operable to provide a selected length, offset, or size between the engaging member and the femoral head.

13. A prosthesis for replacing a first portion of an anatomy, comprising:
a stem member operable to extend into the first portion of the anatomy;
a connection portion formed relative to the stem member;
an articulation member operable to engage the connection portion to hold the articulation member relative to the stem member;
an articulation surface defined by an exterior surface of the articulation member operable to contact a second portion of the anatomy; and
a guard surface defined relative to the articulation surface operable to hold a third portion of the anatomy at a selected position relative to the articulation member.

14. The prosthesis of claim 13, wherein the articulation member is sized and configured to replace a femoral head of the first portion of the anatomy;
wherein the guard surface is operable to engage a PSOAS muscle, tendon, or combinations thereof.

15. The prosthesis of claim 13, further comprising:
a guard portion defining the guard surface and operable to be fixed relative to the articulation member;
wherein the guard surface is substantially continuous with the articulation surface.

16. The prosthesis of claim 15, further comprising:
an adapter member;
wherein the adapter member is operable to interconnect the articulation member and the guard portion and further includes a connection region operable to engage the connection portion to hold the articulation member relative to the stem member.

17. The prosthesis of claim 16, further comprising:
a bore defined by the adapter operable to receive a projection extending from the guard portion.

18. The prosthesis of claim 16, wherein the guard portion includes a guard member, and further comprising:
a projecting member extending from the guard member having a length greater than a height and a finger at a terminal end of the projecting member;
wherein the adapter includes an exterior surface having a recess defined therein extending towards a center of the adapter from the exterior surface; and
wherein the projecting member is operable to extend in the recess and the finger is operable to engage a portion of the exterior surface of the adapter to hold the guard portion relative to the adapter member.

19. The prosthesis of claim 16, wherein the guard portion includes a first member and a second member;
wherein the first member includes an exterior surface defining the guard surface extending a first direction and a projection extending a second direction from the guard surface and the second member defines a throughbore; and
wherein the projection extends through the throughbore and engages the second member operable to fix the first member relative to the second member.

20. The prosthesis of claim 16, wherein the adapter defines a first male taper to engage a first female taper defined by the articulation member;
wherein the adapter further defines a second female taper to engage a second male taper defined by the connection portion;
wherein the adapter further defines a third female taper to engage a third male taper defined by the guard portion;
wherein the stem member includes a plurality of stem members and the articulation member includes a plurality of articulation members; and
wherein the adapter is operable to engage each of the stem members and each of the articulation members.

21. A method of replacing a portion of an anatomy; comprising:
providing a stem portion for implanting into a first anatomical portion of the anatomy;
providing an articulation portion extending from the stem portion having an articulation surface;
implanting the stem portion and the articulation portion; and
providing a guard portion extending from the articulation portion, wherein the guard portion engages a second anatomical portion of the anatomy.

22. The method of claim 21, further comprising:
providing a second surface on the articulation portion; and
covering the second surface with the guard;
wherein the guard portion covering the second surface contacts the second anatomical portion so that the second surface is unable to contact the second anatomical portion.

23. The method of claim 22, further comprising:
wherein providing the articulation surface includes substantially defining a sphere; and
providing a passage through a surface of the sphere to receive the at least a portion of the stem portion.

24. The method of claim 22, wherein the portion of a sphere defined by the articulation surface is less than a portion of the sphere defined by the cooperation of the articulation surface and a third surface of the guard.

25. The method of claim 22, further comprising:
after implantation, moving the second anatomical portion with the guard portion so that the second anatomical portion does not substantially engage an edge of the articulation portion.

26. The method of claim 25, wherein implanting the stem member includes:
positioning the stem member into a femoral bone; and
positioning the articulation member relative to the stem member;
wherein moving the second anatomical portion includes moving a PSOAS muscle, tendon, or combinations thereof.

27. The method of claim 21, further comprising:
providing an adapter operable to engage the stem portion and the articulation portion;
wherein the adapter portion is operable to interconnect the articulation portion and the guard portion.

28. The method of claim 27, further comprising:
interconnecting at least the articulation portion, the adapter portion, and the guard portion during an operative procedure.

29. The method of claim 27, further comprising:
selecting an offset, a position of a femoral head relative to a stem, or combinations thereof with the adapter.

30. The method of claim 27, wherein providing a stem and providing an articulation portion includes providing a plurality of stems and a plurality of articulation portions;
wherein the provided adapter is operable to interconnect each of the plurality of stems and each of the plurality of articulation portions.
